SEQUENCE LISTING (4 sequences)

SEQ ID NO: 1 - protein, 271 aa, homo sapiens
VAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 2 - DNA, 813 bp, Homo sapiens
gtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctggggtccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcac

SEQ ID NO: 3 - protein, 300 aa, Homo sapiens
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 4 - DNA, 927 bp, Homo sapiens
  misc_feature (25)..(924): CDS = 25 to 924
gctctccctgctccagcaaggaccatgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgcgcccagtgccccccaggcacctttgtgcagcggccgtgccgccgagacagccccacgacgtgtggcccgtgtccaccgcgccactacacgcagttctggaactacctggagcgctgccgctactgcaacgtcctctgcggggagcgtgaggaggaggcacgggcttgccacgccacccacaaccgtgcctgccgctgccgcaccggcttcttccacgctggtttctgcttggagcacgcatcgtgtccacctggtgccgtgattgccccgggcacccccagccagaacacgcagtgccagccgcccccaggcaccttctcagccagcagctccagctcagagcagtgccagccccaccgcaactgcacggccctgggcctggccctcattgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctgggctccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcactgatcctggccc

(19) United States
(12) Patent Application Publication
(10) Pub. No.: US 2004/0176279 A1
(43) Pub. Date: Sep. 9, 2004

(54) Flint glycoforms

(76) Inventors: Nigel Jenkins, Lausanne (GB); Derrick Ryan Witcher, Fishers, IN (US); Victor John Wroblewski, Carmel, IN (US)

Correspondence Address: Thomas D Webster, Eli Lilly & Company, Patent Division, PO Box 6288, Indianapolis, IN, 46206-6288, US

(21) Appl. No.: 10/466,786

(22) PCT Filed: Jan. 18, 2002
(86) PCT No.: PCT/US02/00510

Related U.S. Application Data
(60) Provisional application No. 60/265,749, filed on Feb. 1, 2001.

(51) Int. Cl. A61K 38/17

(52) U.S. Cl. 514/8

(57) ABSTRACT

The present invention provides FLINT isoforms having an average sialic acid content of 0.5, 1.0, 1.5, 2.0, 2.5, 3.0, 3.5, 4.0 or greater than 4.0 per molecule of FLINT. Also provided are mixtures of said isoforms, and methods for making said isoforms.

### BACKGROUND OF THE INVENTION

FLINT is a glycoprotein involved in regulating apoptosis. A number of tumor necrosis factor receptor proteins (“TNFR proteins”) have been isolated in recent years, having many potent biological effects. Loss of normal activity of these proteins has been implicated in a number of disease states.

Increased activation of the Fas-FasL signal transduction pathway is implicated in a number of pathological conditions, including runaway apoptosis (Kondo et al., Nature Medicine 3(4):409-413 (1997); Galle et al., J. Exp. Med. 182:1223-1230 (1995)), and inflammatory disease resulting from neutrophil activation (Miwa et al.,. Nature Medicine 4:1287 (1998)). “Runaway apoptosis” is a level of apoptosis greater than normal, or apoptosis occurring at an inappropriate time. Pathological conditions caused by runaway apoptosis include, for example, organ failure in the liver, kidneys and pancreas. Inflammatory diseases associated with excessive neutrophil activation include sepsis, ARDS, SIRS and MODS.

One particular TNFR homologue, referred to herein as “Fas Ligand Inhibitory Protein,” or “FLINT”, binds Fas Ligand (FasL) thereby preventing the interaction of FasL with Fas. FLINT also binds the ligand known as LIGHT to prevent the interaction of LIGHT with receptor LTBR, an otherwise initiating step in a second, independent apoptotic pathway.

Compounds such as FLINT can be used to treat or prevent diseases or conditions in mammals, including humans, that clinically may correlate with either, or both, of the binding interactions involving Fas-FasL and LIGHT-LTBR.

Many eucaryotic secretory proteins including FLINT are modified with one or more oligosaccharide groups (See PCT applications WO 00/58466, WO 00/58465, and WO 99/50413). The state of glycosylation of such proteins can dramatically affect their physical properties and also be important for stability, secretion, and subcellular localization. Moreover, proper glycosylation can be essential for biological activity. In fact, some genes from eucaryotic organisms, when expressed in bacteria (e.g., E. coli) yield proteins that have little or no activity by virtue of their lack of glycosylation.

Glycosylation occurs at specific locations along the polypeptide backbone and is usually of two types: O-linked oligosaccharides are attached to serine or threonine residues, while N-linked oligosaccharides are attached to asparagine residues when part of the consensus sequence Asn-X-Ser/Thr, where X can be any amino acid except proline.

Additionally, the structure and composition of N-linked and O-linked oligosaccharides differ. One type of sugar that is commonly found on N-linked and O-linked oligosaccharides is N-acetylneuraminic acid (hereafter referred to as sialic acid). Sialic acid is usually the terminal residue on both N-linked and O-linked oligosaccharides and, by virtue of its negative charge, may confer acidic properties on a glycoprotein.

FLINT has an N-linked glycosylation site at Asn 173 of SEQ ID NO:3 (Asn 144 of SEQ ID NO:1), and O-linked glycosylation sites at Thr 203 of SEQ ID NO:3 (alternatively, Thr 174 of SEQ ID NO:1); and Thr 245 of SEQ ID NO:3 (alternatively, Thr 216 of SEQ ID NO:1). Under ordinary recombinant production, the O-linked site at Thr 216 of SEQ ID NO:1 is substantially less glycosylated than at Thr 174 of SEQ ID NO:1.

The sialic acid content of FLINT and its effect on the PK profile of the molecule has heretofore not been defined. Shown herein is evidence that the in vivo clearance of FLINT from serum is dependent on the level of sialic acid content. Under-sialylated FLINT is cleared more rapidly from the serum of primates than FLINT with relatively greater sialylation content. This may be explained by clearance of asialylated FLINT, or low sialylated FLINT, from the circulation upon interaction with certain hepatic receptors, for example, the hepatic asialoglycoprotein binding protein (cf. Morrell et al. J. Biol. Chem. 243, 155 (1968); Briggs, et al. Am. J. Physiol. 227, 1385 (1974); Ashwell et al. Methods Enzymol. 50, 287 (1978)). Achieving enhanced levels of sialylation and slower clearance in vivo is therefore expected to improve the therapeutic utility of FLINT.

It is an object of the present invention to provide isoforms of FLINT having enhanced sialic acid content. Pharmaceutical compositions comprising such molecules provide FLINT compositions with slower clearance time in vivo, and enhanced therapeutic value.

### SUMMARY OF THE INVENTION

The subject invention relates to FLINT sialic acid isoforms. Also provided are methods of preparing FLINT isoforms and pharmaceutically acceptable compositions comprising FLINT isoforms. This invention also relates to therapeutic methods comprising administering a therapeutically effective amount of these FLINT compositions to treat and/or prevent diseases or conditions in mammals including humans.

The subject invention relates further to methods of preparing FLINT isoforms comprising subjecting material containing FLINT to ion exchange chromatography, liquid chromatography, or chromatofocusing as well as methods to enhance sialylation of FLINT comprising use of enzymatic processes.

### DETAILED DESCRIPTION OF THE INVENTION

SEQ ID NO:1—Mature human FLINT, i.e. native FLINT minus the leader sequence.

SEQ ID NO:2—Nucleic acid/cDNA encoding mature human FLINT.

SEQ ID NO:3—Native human FLINT.

SEQ ID NO:4—Nucleic acid encoding human FLINT.

The term “analog” or “FLINT analog” is used herein specifically to mean a FLINT sequence variant having one or more amino acid sequence changes in SEQ ID NO:1 or SEQ ID NO:3, e.g. substitution, addition, deletion, said changes producing analogs that are protease-resistant between positions 218 and 219 of SEQ ID NO:1 (positions 247 and 248 of SEQ ID NO:3). Protease-resistant analogs such as R218Q may also comprise additional N-linked glycosylation sites. For example, analog R34N, D36T, R218Q contains one additional site; and R34N, D36T, D194N, S196T, R218Q; contains two additional glycosylation sites. Said analogs retain the biological activity of FLINT.

As used herein, “average sialic acid content” refers to a quantitative measure of the sialic acid content of a FLINT sample preparation, expressed as the mole fraction of sialic acid per mole of FLINT. The term is useful for comparing different preparations of FLINT. FLINT preparations may comprise multiple FLINT isoforms, for example, 0, 1, 2, 3, 4, 5, 6, 7, 8, or more sialic acids per molecule of FLINT.

The term “native FLINT” refers to SEQ ID NO:3.

The term “mature FLINT” refers to SEQ ID NO:1.

The description herein of FLINT isoforms is referenced to either SEQ ID NO:1 or SEQ ID NO:3. Both the mature and native forms of FLINT are intended to be covered by the invention.

The term “FLINT” encompasses native, mature, and protease-resistant analogs of FLINT. FLINT is a member of the TNFR family having the ability to inhibit Fas-FasL induced apoptosis, in vitro and in vivo.

“FLINT isoform” refers to sialic acid variants of FLINT. Native FLINT can occur with and accommodate 0, 1, 2, 3, 4, 5, or 6 sialic acids per molecule of FLINT, based on one N-linked site at Asn 144 of SEQ ID NO:1, and one O-linked site at Thr 174 of SEQ ID NO:1. A second O-linked site, at Thr 216 of SEQ ID NO:1, is substantially less glycosylated than the site at Thr 174. Protease-resistant analog R218Q can occur with 0, 1, 2, 3, 4, 5, 6, 7, or 8 sialic acids per molecule of protein, based on one N-linked site at Asn 144 of SEQ ID NO:1, one O-linked site at Thr 174 and one O-linked site at Thr 216 of SEQ ID NO:1. O-linked glycosylation is substantially enhanced at position 216 of R218Q (SEQ ID NO:1) when compared with native FLINT. The degree of sialylation will depend on the host cell and growth conditions. Sialylation can be enhanced in vitro using an enzymatic process described later in this disclosure.

The term “N-glycosylated polypeptide” refers to polypeptides having one or more NXS/T motifs in which the nitrogen atom in the side chain amide of the asparagine is covalently bonded to a glycosyl group. “X” refers to any naturally occurring amino acid residue except proline. The “naturally occurring amino acids” are glycine, alanine, valine, leucine, isoleucine, proline, serine, threonine, cysteine, methionine, lysine, arganine, glutamic acid, asparatic acid, glutamine, asparagine, phenylalanine, histidine, tyrosine and tryptophan. N-Glycosylated proteins are optionally O-glycosylated.

The term “O-glycosyled polypeptide” refers to polypeptides having one or more serines and/or threonine in which the oxygen atom in the side chain is covalently bonded to a glycosyl group. O-Glycosylated proteins are optionally N-glycosylated.

The term “protease-resistant” refers to a FLINT analog that, when compared with FLINT, is more resistant to proteolysis between residues 218 and 219 of SEQ ID NO:1 (See PCT International Application WO 00/58466). Protease resistant analogs differ from FLINT by one or more amino acid substitutions, deletions, inversions, additions, and/or changes in glycosylation sites, or patterns, as compared with or against native FLINT, or mature FLINT. Preferably sequence changes occur in the region from about position 214 through position 222 of SEQ ID NO:1.

The nucleotide and amino acid abbreviations used herein are those accepted in the art and by the United States Patent and Trademark Office, as set forth in 37 C.F.R. 1.822 (b)(2).

The present invention relates to sialic acid isoforms of FLINT including isoforms of native FLINT (SEQ ID NO:1 and/or SEQ ID NO:3), and isoforms of protease-resistant analogs of FLINT in which one or more amino acids are substituted, deleted, or added to native FLINT to produce protease resistance and, in some cases, add additional sites for glycosylation.

Protease-resistant analogs comprise one or more amino acid changes in the region defined by positions 214 through 222 of SEQ ID NO:1. Specific examples include single amino acid substitutions for Arg at position 218, to Gln, Glu, Ala, Gly, Ser, Val, or Tyr. Other examples include one or more substitutions in the window 214 through 222 of SEQ ID NO:1 including, Gly214 to any naturally occurring amino acid, Pro215 to any naturally occurring amino acid, Thr216 to any naturally occurring amino acid, Pro217 to any naturally occurring amino acid, Arg218 to any naturally occurring amino acid, Ala219 to any naturally occurring amino acid, Gly220 to any naturally occurring amino acid, Arg221 to any naturally occurring amino acid, Ala222 to any naturally occurring amino acid.

Other substitutions include Gly214 to a positively charged amino acid defined as His, Arg or Lys, Pro215 to a positively charged amino acid, Thr216 to a positively charged amino acid, Pro217 to a positively charged amino acid, Arg218 to a positively charged amino acid, Ala219 to a positively charged amino acid, Gly220 to a positively charged amino acid, Arg221 to a positively charged amino acid, Ala222 to a positively charged amino acid.

Other substitutions include Gly214 to a negatively charged amino acid defined as Asp or Glu, Pro215 to a negatively charged amino acid, Thr216 to a negatively charged amino acid, Pro217 to a negatively charged amino acid, Arg218 to a negatively charged amino acid, Ala219 to a negatively charged amino acid, Gly220 to a negatively charged amino acid, Arg221 to a negatively charged amino acid, Ala222 to a negatively charged amino acid.

Other substitutions include Gly214 to a polar uncharged amino acid defined as Cys,Thr, Ser, Gly, Asn, Gln,Tyr, Pro215 to a polar uncharged amino acid, Thr216 to a polar uncharged amino acid, Pro217 to a polar uncharged amino acid, Arg218 to a polar uncharged amino acid, Ala219 to a polar uncharged amino acid, Gly220 to a polar uncharged amino acid, Arg221 to a polar uncharged amino acid, Ala222 to a polar uncharged amino acid.

Other substitutions include Gly2l4 to a nonpolar amino acid defined as Ala, Pro, Met, Leu, Ile, Val, Phe, Trp, Pro215 to a nonpolar amino acid, Thr216 to a nonpolar amino acid, Pro217 to a nonpolar amino acid, Arg218 to a nonpolar amino acid, Ala219 to a nonpolar amino acid, Gly220 to a nonpolar amino acid, Arg221 to a nonpolar amino acid, Ala222 to a nonpolar amino acid.

Other substitutions include multiple changes that may add one or more additional N-linked glycosylation sites to a protease-resistant FLINT. For example, Arg34 to Asn, Asp36 to Thr, Arg218 to Gln, Glu, Ala, Gly, Ser, Val or Tyr; Arg34 to Asn, Asp36 to Thr, Asp194 to Asn, Ser196 to Thr, Arg218 to Gln, Glu, Ala, Gly, Ser, Val, or Tyr; or Arg34 to Asn, Asp36 to Thr, Arg218 to any amino acid not Arg, any positively charged amino acid not Arg, any negatively charged amino acid not Arg, any polar uncharged amino acid not Arg, any nonpolar amino acid not Arg, or an amino acid that is Glu, Gln, Ala, Gly, Ser, Val, or Tyr; Arg34 to Asn, Asp36 to Thr, Asp194 to Asn, Ser196 to Thr, Arg218 to any amino acid not Arg, any positively charged amino acid not Arg, any negatively charged amino acid not Arg, any polar uncharged amino acid not Arg, any nonpolar amino acid not Arg, an amino acid that is Glu, Gln, Ala, Gly, Ser, Val, or Tyr; or Ser132 to Asn, Arg218 to any amino acid not Arg, any positively charged amino acid not Arg, any negatively charged amino acid not Arg, any polar uncharged amino acid not Arg, any nonpolar amino acid not Arg, an amino acid that is Glu, Gln, Ala, Gly, Ser, Val, or Tyr.

According to the present invention, FLINT isoforms can be separated by a variety of techniques including isoelectric focusing (IEF). When placed in a pH gradient and subjected to an electric field, proteins will migrate to the point at which they have no net charge. This is the isoelectric point (pI) of the protein. Each distinct band observed on IEF represents molecules that have a particular pI and therefore the same overall charge, termed an isoform. The term “FLINT isoform” as used herein refers to FLINT preparations having about the same pI, as measured by any suitable technique, and the same amino acid sequence.

Other means for separating FLINT isoforms include fractionation over an ion exchange column. Preferably isoforms are separated by liquid chromatography.

In a preferred embodiment, FLINT is expressed recombinantly in a non-human eucaryotic host cell (i.e. “recombinant FLINT”). Recombinant FLINT is advantageously produced and purified according to the procedures described in commonly owned PCT applications WO 00/58466, WO 00/58465, and WO 99/50413, hereby incorporated by reference. For example, FLINT cDNA was incorporated into vector pcDNA3DHFR which provides the CMV promoter to drive FLINT gene expression and DHFR selection. DG44-C.B4 CHO cells were transfected with linearized vector by electroporation. For non-selective propagation, cells were grown in Ex-Cell 302 Medium (JRH BioSciences), 1×HT Supplement (GibcoBRL), 1×dextran sulfate (Sigma) and 6 mM L-glutamine (GibcoBRL). For selective growth, cells were placed in Ex-Cell 302 Medium (JRH BioSciences), 1×HT Supplement (GibcoBRL), 1× dextran sulfate (Sigma), 6 mM L-glutamine (GibcoBRL), and Methotrexate (20 mM stock, USP).

After electroporation cells were placed in non-selective growth medium to recover for 72 hours. Plating was done using 96 well dishes. The cells were plated at various cell densities, and under various levels of methotrexate (MTX) selective pressure. When colony formation was visible the plates were screened by ELISA. The wells were moved into 24 well dishes and expanded to generate enough cells for a full expression study evaluation.

Master wells were amplified at different levels of methotrexate. All master wells showed increased levels of expression at the end of the amplification step. Two master wells were cloned using FACS cloning.

Discrete isoforms of recombinant FLINT (SEQ ID NO:3) correspond to FLINT molecules having from 1-8 sialic acids. Increasing the number of sialic acid residues per molecule of FLINT has the effect of slowing the clearance of FLINT in vivo.

As demonstrated herein, the in vivo clearance of FLINT and protease-resistant analogs, such as R218Q (Arg to Gln at position 218 of SEQ ID NO:1), correlates with sialic acid content (See Examples 3 and 4). Specifically, FLINT having greater sialic acid content, for example, greater average sialic acid content, is cleared more slowly in primates in vivo than the corresponding FLINT having lower sialic acid content. Native FLINT possesses one N-linked site and one O-linked site, whereas the R218Q analog possess one N-linked site and two O-linked sites, the O-linked site additional to native FLINT in R218Q occurs at position 216 of SEQ ID NO:1.

FLINT Isoforms

The subject invention provides compositions of FLINT isoforms. The sialylation pattern of specific isoforms of FLINT may vary depending upon the particular host cell and growth conditions used in production. In a preferred embodiment, the invention relates to an FLINT isoform composition having an average sialic acid content of about 0.5 sialic acid residues per molecule of FLINT; alternatively, an average of about 1.0 sialic acids per molecule of FLINT; alternatively, an average of about 1.5 sialic acids per molecule of FLINT; alternatively, an average of about 2.0 sialic acids per molecule of FLINT; alternatively, an average of about 2.5 sialic acids per molecule of FLINT; alternatively, an average of about 3.0 sialic acids per molecule of FLINT; alternatively, an average of about 3.5 sialic acids per molecule of FLINT; alternatively, an average of about 4.0 sialic acids per molecule of FLINT; an average of about 4.5 sialic acids per molecule of FLINT; an average of about 5.0 sialic acids per molecule of FLINT; an average of about 5.5 sialic acids per molecule of FLINT; an average of about 6.0 sialic acids per molecule of FLINT; alternatively, an average greater than about 6.0 sialic acids per molecule of FLINT.

The compositions of the invention, defined in terms of average sialic acid content, may comprise different FLINT isoforms, each of which may have a different sialic acid content or profile. For example, an N-linked site theoretically could accommodate 0, 1, 2, 3, or 4 sialic acids per site; an O-linked site could theoretically accommodate 0, 1, or 2 sialic acids per site. Consider a composition of native FLINT having an average sialic acid content of about 1 sialic acids per mole of FLINT. A composition falling within the scope of this description could comprise species that are on average sialylated once at the N-linked site, or, alternatively, once at the O-linked site. Other examples falling within the scope of the invention will be understood by the skilled artisan to include multiple sialylations of a fraction of FLINT molecules in a composition. The compositions of the invention are intended to cover any and all such combinations of isoforms.

The invention also provides methods for preparing FLINT isoform compositions. These methods include isolation of isoforms by techniques such as preparative isoelectric focusing, or ion exchange chromatography, or chromatofocusing.

In general, ion exchange chromatography and chromatofocusing involve application of either conditioned medium containing FLINT, or purified material, to a column resin under conditions that permit binding of some or all of the FLINT isoforms to the resin. It is preferable to apply the protein to the column at about pH 5. After washing the column with buffer at about pH 5, FLINT isoforms that remain bound on the ion exchange column are eluted by increasing the salt concentration of the buffer. For chromatofocusing, isoforms are eluted from the column by a gradient of decreasing pH, or by washing the column with a high concentration of salt.

FLINT molecules have N-linked or O-linked oligosaccharide structures that can limit the sialic acid content of the molecule. For example, tetra-antennary (four-branched) N-linked oligosaccharides provide four possible sites for sialic acid attachment, while bi- and triantennary oligosaccharide chains, which can substitute for the tetra-antennary form at asparagine-linked sites, commonly have only two or three sialic acids attached. O-linked oligosaccharides commonly provide two sites for sialic acid attachment.

Thus, FLINT molecules can accommodate a total of 8 sialic acid residues provided the single N-linked oligosaccharides is tetra-antennary. The N-linked oligosaccharides of FLINT contain sialic acid in both an α-2,3 and an α-2,6 linkage to galactose (Takeuchi et al. J. Biol. Chem. 263, 3657(1988)). Typically the sialic acid in the α-2,3 linkage is added to galactose on the mannose α-1,6 branch, and the sialic acid in the α-2,6 linkage is added to the galactose on the mannose α-1,3 branch. The enzymes that add-these sialic acids β-galactoside α-2,3 sialyltransferase and β-galactoside α-2,6 sialyltransferase) are most efficient at adding sialic acid to the mannose α-1,6 and mannose α-1,3 branches, respectively.

Mammalian cell cultures may be screened for cells that preferentially add tetra-antennary chains to recombinant FLINT, thereby maximizing the number of sites for sialic acid attachment. Dihydrofolate reductase (DHFR) deficient Chinese Hamster Ovary (CHO) cells are commonly used for the production of recombinant glycoproteins including recombinant FLINT. These cells do not express the enzyme β-galactoside α-2,6 sialyltransferase, and therefore do not add sialic acid in the α-2,6 linkage to N-linked oligosaccharides of glycoproteins produced in these cells. (Mutsaers et al. Eur. J. Biochem. 156, 651 (1986); Takeuchi et al. J. Chromatogr. 400, 207 (1987)). Consequently, recombinant FLINT produced in CHO cells lacks sialic acid in the 2,6 linkage to galactose (Sasaki et al. (1987), supra; Takeuchi et al. (1987), supra). Therefore, in one embodiment of the invention, FLINT isoforms are made in CHO cells that are transfected with a functional β-galactoside α-2,6 sialyltransferase gene to give incorporation of sialic acid in α-2,6 linkage to galactose. See Lee et al. J. Biol. Chem. 264, 13848 (1989), hereby incorporated by reference, for a disclosure of techniques for creating modified CHO cells, or other mammalian host cells.

In Vitro Enhancement of Sialylation of FLINT

Also contemplated by the present invention is a method for enhancing the sialylation of FLINT by enzymatic modification in vitro.

The circulatory lifetime of glycoproteins such as FLINT in blood is highly dependent on the composition and structure of N-linked oligosaccharides. In general, maximal plasma half-life of a glycoprotein requires that its N-linked carbohydrate groups terminate in the sequence NeuAcGalGlcNAc. Without a terminal sialic acid residue (NeuAc), a glycoprotein is rapidly cleared from the blood by receptors that recognize the exposed Gal residues. For this reason, ensuring high sialylation of therapeutic proteins such as FLINT is important for commercial development.

Although much is known about the complexity of carbohydrate structures on glycoproteins, attempts to specify post-translational glycosylation in cultured cells have not kept pace with advances in technology for gene expression, and therefore, incomplete glycosylation of secreted recombinant glycoproteins, including FLINT, is common. One solution to this problem is to use isolated glycosyltransferases to complete carbohydrate chains in vitro.

Optimal glycosylation can be difficult to achieve using mammalian cell culture systems. Under conditions of large scale growth, overproduction of a protein backbone comprising a glycoprotien can exceed the host cells capacity to achieve full sialylation.

A method of the present invention comprises use of sialyltransferase to add sialic acids to an acceptor site(s) on FLINT, preferably said site(s) having a galactosyl unit. The method for enhancing sialylation of FLINT follows that disclosed in U.S. Pat. No. 6,030,815, herein incorporated by reference. Essentially, the method comprises the steps of adding sialyltransferase to a sample of FLINT and a catalytic amount of a CMP-sialic acid synthetase, a sialic acid, CTP, and a soluble divalent metal cation, including Mn⁺², Mg⁺², Ca⁺², Co⁺², and Zn⁺². Preferably, the divalent ion concentration is maintained between 2 mM and 75 mM. Alternatively, the reaction may further comprise a CMP-sialic acid recycling system, as disclosed in U.S. Pat. No. 6,030,815. A commercially available system (GlycoAdvance®) for carrying out the reactions is available from Neose Technologies, Inc. (Horsham, Pa.).

In another embodiment of the invention, FLINT is produced recombinantly in a suitable mammalian cell type, for example, CHO cells, by transfection with a suitable vector for expressing FLINT. Culture supernatants containing FLINT are concentrated and processed using the GlycoAdvance® system, or comparable commercial or non-commercial system. FLINT having enhanced sialylation is recovered using standard purification techniques.

Also comprehended by the invention are pharmaceutical compositions comprising a therapeutically effective amount of a FLINT isoform, having an average sialic acid content of about 0.5 sialic acids per molecule of FLINT, alternatively, an average of about 1.0 sialic acids per molecule of FLINT; alternatively, an average of about 1.5 sialic acids per molecule of FLINT; alternatively, an average of about 2.0 sialic acids per molecule of FLINT; alternatively, an average of about 2.5 sialic acids per molecule of FLINT; alternatively, an average of about 3.0 sialic acids per molecule of FLINT; alternatively, an average of about 3.5 sialic acids per molecule of FLINT; alternatively, an average of about 4.0 sialic acids per molecule of FLINT; alternatively, an average of about 4.5 sialic acids per molecule of FLINT; alternatively, an average of about 5.0 sialic acids per molecule of FLINT; alternatively, an average of about 5.5 sialic acids per molecule of FLINT; alternatively, an average of about 6.0 sialic acids per molecule of FLINT; alternatively, an average greater than about 6.0 sialic acids per molecule of FLINT, together with a suitable diluent, adjuvant and/or carrier useful in therapeutic applications. A “therapeutically effective amount” as used herein refers to that amount which provides therapeutic effect for a given condition and administration regimen. The administration of FLINT isoforms is preferably by the intravenous route.

Therapeutic Applications

The clinical utility for the FLINT isoforms of the invention is expected to be substantial. FLINT inhibits the binding of Fas to FasL and LIGHT to LTBR and TR2/HVEM receptors, and can be used to treat or prevent a disease and/or condition that may be associated with such binding.

Many diseases and/or conditions involving FasL/Fas are potentially amenable to therapy with FLINT. Examples of suitable diseases and/or conditions include the following.

Inflammatory/autoimmune diseases—Rheumatoid arthritis, inflammatory bowel disease, graft-versus-host disease, insulin-dependent diabetes, SIRS/sepsis/MODS, pancreatitis, psoriasis, multiple sclerosis, Hashimoto's thyroiditis, Grave's disease, transplant rejection, SLE, autoimmune gastritis, fibrosing lung disease.

Infectious diseases—HIV-induced lymphopenia, fulminant viral hepatitis B/C, chronic hepatitis/cirrhosis, H. pylori-associated ulceration.

Ischemia/Re-perfusion conditions—Acute coronary syndrome, acute myocardial infarction, congestive heart failure, atherosclerosis, acute cerebral ischemia/infarction, brain/spinal cord trauma, organ preservation during transplant

Other treatments include cytoprotection during cancer treatment, adjuvant to chemotherapy, Alzheimer's, chronic glomerulonephritis, osteoporosis, TTP/HUS, aplastic anemia, myelodysplasia. Also of interest are treatment and prevention of acute lung injury (ALI)/acute respiratory distress syndrome (ARDS); Ulcerative colitis; and Crohn's disease.

Other diseases for which FLINT isoforms are therapeutically useful include rheumatoid arthritis (Elliott et al., Lancet 344:1105-10 (1994)), fibroproliferative lung disease, fibrotic lung disease, HIV (Dockrell et al., J. Clin. Invest. 101:2394-2405 (1998)), Ischemia (Sakurai et al. 1998 Brain Res 797:23-28), Brain trauma/injury (Ertel et al. 1997 J Neuroimmunol 80:93.-6), chronic renal failure (Schelling et al. 1998 Lab Invest 78:813-824), Graft-vs-Host Disease (GVHD) (Hattori et al. 1998 Blood 11:4051-4055), Cutaneous inflammation (Orteu at al. 1998 J Immunol 161:1619-1629), Vascular leak syndrome (Rafi et al. 1998 J Immunol 161:3077-3086), Helicobacter pylori infection (Rudi et al. 1998 J Clin Invest 102:1506-1514), Goiter (Tamura et al. 1998 Endocrinology 139:3646-3653), Atherosclerosis (Sata and Walsh, 1998 J Clin Invest 102:1682-1689), IDDM (Itoh et al. 1997 J Exp Med 186:613-618), Osteoporosis (Jilka et al. 1998 J Bone Min Res 13:793-802), Crohn's Disease (van Dullemen et al. 1995 Gastroenterology 109:129-35), organ preservation and transplant (graft) rejection (Lau et al. 1996 Science 273:109-112), Sepsis (Faist and Kim. 1998 New Horizons 6:S97-102), Pancreatitis (Neoptolemos et al. 1998 Gut 42:886-91), Cancer (melanoma, colon and esophageal) (Bennett et al. 1998 J Immunol 160:5669-5675), Autoimmune disease (IBD, psoriasis, Down's Syndrome (Seidi et al., Neuroscience Lett:. 260:9 (1999), multiple sclerosis (D'Souza et al. 1996 J Exp Med 184:2361-70), Alzheimer's Disease; End-stage renal disease (ESRD); mononulceosis; EBV; Herpes; antibody dependent sytotoxicity; hemolytic and hypercoagulation disorders such as vascular bleeds, DIC (disseminated intervascular coagulation), eclampsia, HELLP (preeclampsia complicated by thrombocytopenia, hemolysis and disturbed liver function), HITS (heparin induced thromobcytopenia), HUS (hemolytic uremic syndrome), and preeclampsia; hematopoeitic disorders such as aplastic anemia, thrombocytopenia (TTP) and myelodysplasia; and hemolytic fever caused, for example, by E.bola.

In the case of organ preservation in preparation for harvesting, for instance, FLINT is useful prophylactically to prevent the apoptosis associated with ischemia reperfusion injury to the organ once it is removed from the donor. Suitable media for this purpose are known, for example, the media disclosed in EP 0356367 A2. The method may also include treating the transplant recipient with FLINT prior to and/or after the transplant surgery.

There is evidence that ARDS may be mediated by soluble FasL/Fas interaction in humans (Matute-Bello et al., J. Immunol. 163, 2217-2225, 1999). FLINT, by binding to FasL, could inhibit FasL-mediated apoptosis of pneumocytes and/or endothelial cells, thus inhibiting or preventing the progression from acute inflammatory insult to ALI, and from ALI to ARDS.

Therefore, in another embodiment, the present invention relates to the use of FLINT to inhibit and/or treat ALI and/or ARDS comprising the administration of a therapeutically effective amount of FLINT to a person in need thereof.

In another embodiment, the present invention relates to the use of FLINT analog to treat and/or inhibit chronic obstructive pulmonary disease (COPD) in a patient in need thereof by administering a therapeutically effective amount of FLINT.

In another embodiment the present invention relates to the use of FLINT to inhibit and/or treat pulmonary fibrosis (PF). For example, FLINT can be administered acutely at the time of an inflammatory insult to the lung (e.g. during bleomycin treatment) to prevent PF from occurring.

A “subject” is a mammal in need of treatment, preferably a human, but can also be an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like).

An “effective amount” of FLINT isoform is an amount which results in a sufficient inhibition of one or more processes mediated by the binding of Fas to Fas Ligand or LIGHT to LTBR and/or TR2/HVEM so as to achieve a desired therapeutic or prophylactic effect in a subject with a disease or condition that may be associated with aberrant Fas/FasL binding and/or LIGHT mediated binding. Alternatively, an “effective amount” of FLINT isoform is a quantity sufficient to achieve a desired therapeutic and/or prophylactic effect in a subject with inflammation caused by FasL-induced neutrophil activation or any of the other aforementioned diseases associated with aberrant FasL activity.

A “desired therapeutic and/or prophylactic effect” in a subject with a disease or condition includes the amelioration of symptoms, or delay in onset of symptoms, associated with such disease. Alternatively, a “desired therapeutic and/or prophylactic effect” includes an increased survival rate or increased longevity for the subject with the disease.

The amount of FLINT isoform administered to the individual will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors.

As a general proposition, the total pharmaceutically effective amount of the FLINT isoform molecules of the present invention administered parenterally per dose will be in the range of about 1 μg/kg/day to 10 mg/kg/day of patient body weight, particularly 2 mg/kg/day to 8 mg/kg/day, more particularly 2 mg/kg/day to 4 mg/kg/day, even more particularly 2.2 mg/kg/day to 3.3 mg/kg/day, and finally 2.5 mg/kg/day, although, as noted above, this will be subject to therapeutic discretion. More preferably, this dose is at least 0.01 mg/kg/day. If given continuously, the FLINT isoform of the present invention are typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the FLINT isoform molecules of the present invention may be administered orally, rectally, intracranially, parenterally, intracisternally, intravaginally, intraperitoneally, topically (as by powders, ointments, drops or transdermal patch), transdermally, intrathecally, bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein includes, but is not limited to, modes of administration which include intravenous, intramuscular, intraperitoneal, intrasternal, subcutaneous and intraarticular injection, infusion and implants comprising FLINT isoforms.

The FLINT isoforms of the present invention are also suitably administered by sustained-release systems. Suitable examples of sustained-release compositions include semi-permeable polymer matrices in the form of shaped articles, e.g., films, or microcapsules. Sustained-release matrices include polylactides (U.S. Pat. No. 3,773.919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Other sustained-release compositions also include liposomally entrapped FLINT. Such liposomes are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EDP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal TNFR polypeptide therapy.

For parenteral administration, the FLINT isoforms of the present invention are formulated generally by mixing at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

The FLINT isoforms of the present invention are typically formulated in suitable vehicles at a concentration of about 0.1 mg/ml to 100 mg/ml, preferably 1-10 mg/ml, at a pH of about 3 to 8. It will be understood that the use of certain excipients, carriers, or stabilizers will result in the formation of salts of the FLINT molecules of the present invention.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the FLINT isoforms of the present invention may be employed in conjunction with other therapeutic compounds.

The following examples are offered to more fully illustrate the invention, but are not to be construed as limiting the scope thereof.

### EXAMPLE 1

### Isolation of Recombinant FLINT Isoforms

A bicistronic expression vector was constructed by inserting into mammalian expression vector pGTD (Gerlitz, B. et al., 1993, Biochemical Journal 295:131) a PCR fragment encoding an “internal ribosome entry site”/enhanced green fluorescent polypeptide (IRES/eGFP). The new vector, designated pIG3, contains the following elements: the E1a-responsive GBMT promoter (D. T. Berg et al., 1993 BioTechniques 14:972; D. T. Berg et al., 1992 Nucleic Acids Research 20:5485); a multiple cloning site (MCS); the IRES sequence from encephalomyocarditis virus (EMCV); the eGFP coding sequence (Cormack, et al., 1996 Gene 173:33, Clontech); the SV40 small “t” antigen splice site/poly-adenylation sequences; the SV40 early promoter and origin of replication; the murine dihydrofolate reductase (dhfr) coding sequence; and the ampicillin resistance gene and origin of replication from pBR322.

Based on the human FLINT cDNA sequence (e.g. SEQ ID NO:3), forward and reverse PCR primers were synthesized bearing BclI restriction sites at their respective 5′ ends. These primers were used to amplify the FLINT cDNA. The FLINT cDNA orientation and nucleotide sequence was confirmed by restriction digest and double stranded sequencing of the insert. The approximately 900 base pair amplified FLINT PCR product was digested with restriction endonucleases NheI and XbaI, respectively, to generate a fragment bearing NheI and XbaI sticky ends. This fragment was subsequently ligated into a unique XbaI site of pIG3 to generate recombinant plasmid pIG3-FLINT.

The recombinant pIG3-FLINT plasmid carries the FLINT gene and encodes resistance to methotrexate. AV12 RGT18 cells are transfected using a calcium phosphate procedure with the recombinant pIG vector. Cells resistant to 250 nM methotrexate were selected and pooled. The pool of resistant clones was subjected to fluorescence-assisted cell sorting (FACS), and cells having fluorescence values in the top 5% were sorted into a pool, and as single cells. High fluorescence pools were subjected to two successive sorting cycles. Pools and individual clones from the first and second cycles were analyzed for FLINT production by ELISA. Pools or clones expressing FLINT at the highest level were used for scale-up and FLINT purification.

Large scale production of FLINT was carried out by growing stable clones of AV12 RGT 18 cells transfected with pIG3-FLINT in several 10 liter spinners. After reaching confluence, cells were further incubated for 2-3 more days to secrete maximum amount of FLINT into the growth medium. Medium containing FLINT was adjusted to 0.1% CHAPS and concentrated in an Amicon ProFlux M12 tangential filtration system to 350 ml. The concentrated media was adjusted to pH 6.0 and passed over a SP Sepharose Fast Flow (Pharmacia, 500 ml) at a flow rate of 7 ml/min. The column was washed with buffer A (20 mM MOPS, 0.1% CHAPS, pH 6.0) until the absorbance (280 nm) returned to baseline and bound polypeptides were eluted with a linear gradient from 0 to 1 M NaCl (in buffer A) developed over four column volumes. Fractions containing FLINT were pooled and passed over Vydac C4 column (100 ml) equilibrated with 0.1% TFA/H₂O at a flow rate of 10 ml/min. This material is passed over a 16/60 Superdex 200 sizing column (Pharmacia) equilibrated with PBS, 0.5 M NaCl, pH 7.4. Fractions containing FLINT are analyzed by SDS-PAGE, and the N-terminal sequence of the purified polypeptide confirmed to be FLINT.

In an alternative purification scheme, concentrated and clarified medium containing FLINT is passed over Blue Sepharose DAC and eluted with 7M urea, 1M NaCl, pH 8. Eluted material is further purified on CG71 reverse phase chromatography and eluted with 35% acetonitrile, 0.6 M NaCl, pH 7.4. The eluted material is purified further on SP650M cation exchange with elution in 30% acetonitrile, pH 2.5. Following this step, eluted material is subjected to solvent exchange by TFF into buffer for bulk freezer storage.

FLINT has one N-linked glycosylation site at Asn144 of SEQ ID NO:1. To characterize the oligosaccharide structure of FLINT produced in AV12 cells, samples of intact FLINT, and FLINT that had been treated with neuraminidase and HEXase II to release terminal GalNAc and GlcNAc residues (desialylated FLINT), were analyzed by capillary HPLC/ESI-MS. The oligosaccharides released by this treatment were labeled with 2-aminobenzamide and analyzed by weak anion exchange (WAE) HPLC and by LC mass spectrophotometry (LC/MS).

### EXAMPLE 2

### Oligosaccharide Profile of FLINT by HPLC/ESI-MS and Fluorescence HPLC

FLINT has one N-glycosylation site. Recombinant FLINT used for the analysis was expressed in AV12 cells, and purified as in Example 1. Intact FLINT was directly analyzed by a capillary HPLC/ESI-MS, or treated by neuraminidase, or HEXase II, followed by HPLC/ESI-MS. The oligosaccharide structures were calculated based on the obtained masses and the expected FLINT peptide backbone mass. Fluorescence labeled FLINT oligosaccharides were fractionated by weak anion exchange (WAE) HPLC. The fractions were collected and identified by LC/MS.

Neuraminidase, HEXase II Treatment of FLINT.

Ten microliters of a solution containing FLINT (0.43 mg/ml in PBS, 0.5M NaCl) was mixed with 8 uL of 50 mM NaOAc buffer, pH 5.2, and 2 uL of neuraminidase solution (1 unit/mL). The mixture was incubated at 37° C. for 2 hours. Seven microliters of the mixture was used for capillary HPLC/MS analysis and two microliters of HEXase II enzyme solution (Glyko, Inc.) was added to the remaining solution, which was incubated at 37° C. for 3 hours before HPLC/MS analysis.

Weak Anion Exchange (WAE) HPLC of Fluorescence Labeled Oligosaccharides.

A 200 ul Aliquot of thawed FLINT solution containing approximately 0.2 mg of protein was mixed with 60 mg urea, 17.6 ul of 3 M Tris buffer (pH 8.0) and 3 ul of 50 mg/mL dithiothreitol and the mixture was incubated at 37° C. for 10 min. The sample was alkylated by adding 5 ul of 100 mg/mL iodoacetic acid solution and incubating at ambient temperature in the dark for 10 min. Samples were desalted on a disposable gel filtration column and oligosaccharides released by treatment with 1 unit N-glycosidase F solution at 37° C. for 2 hours. The deglycosylated protein was precipitated by adjusting pH with 10% (v/v) acetic acid solution. A 300 ul aliquot of oligosaccharide solution was dried and labeled with 2-aminobenzamide dye. After the excess dye was removed using a P-2 spin column, the labeled-oligosaccharide solution was analyzed by WAE-HPLC with a fluorescence detector.

Desialylation of WAE HPLC Fractions of 2-AB Labeled FLINT Oligosaccharides.

Each of the collected WAE HPLC fractions was transferred into two vials and dried using a centrifugation vacuum system. Fifteen microliters (containing 2.5 munits neuraminidase) of 15 mM NaOAc buffer, pH 5.2 was added to one vial for each fraction. Five microliters of the mixture was used for LC/MS analysis after incubation at room temperature for 10 to 15 hours. For intact oligosaccharide fraction, fifteen microliters of H₂O was added into each vial and 5 ul of the solution was used for. LC/MS analysis.

Capillary HPLC/ESI-MS.

A Beckman System Gold equipped with a Model 126 solvent delivery module was used. The HPLC buffer (A: 0.15% formic acid in H₂O and B: 0.12% formic acid in ACN) was pumped through a T split, in which a Zorbax 300SB C18, 2.1×150 mm column was attached on one exit and a manual injection valve, Vydac capillary column (C18, 0.3×150 mm) and a API UV detector (785A) at the other exit. The HPLC stream from the capillary directly passes to the mass spectrometer through a fused silica transfer line. Beckman solvent deliver system pumped at 0.2 ml/min with the following gradient

| | | | | | | | |
|---|---|---|---|---|---|---|---|
| Time (min) | 0 | 2 | 42 | 43 | 45 | 46 | 57 |
| Buffer B % | 10 | 10 | 45 | 90 | 90 | 10 | 10 |

After the split, about 5-6 ul/min of HPLC stream was diverted to the capillary column. About 2 ug FLINT was injected to capillary column per run. API UV detector was set lambda=214 nm and the data were stored in HP1000. A PE Sciex API III mass spectrometer equipped with an articulated Ionspray source was used in these studies under the condition of CC 1, OR 55 V and ISV 4800 V, Q1 scan from 900 to 1500 or 1000 to 1400, 0.33 or 0.20/step, 2 to 3 ms dwell time and 6 sec/scan. For oligosaccharide fraction analysis, a short solvent gradient program was used.

FLINT has one N-glycosylation site, Asn-144. The theoretical molecular weight of the protein alone is 29736.9 Da. The mass spectrum and reconstructed mass spectrum of intact FLINT reveal molecular weights for the primary signals of 31839, 31883, 31918, 32090, 32131, 32169 Da, which are about 2 kDa higher than the molecular weight of expected protein sequence. These masses are the expected molecular weight of diantennary oligosaccharides. The qualitative analysis of neutral and amino monosaccharide composition indicated that FLINT contains GlcNAc, GlaNAc, Man, Gal and Fuc. The signal at 32131 Da for intact FLINT is consistent with FLINT protein 1-299 with carbohydrate moiety of (NeuAc)2(Hex)4(HexNAc)5(dHex)1 (29736.9+2393.2=32130.1 Da). After desialylation, this signal was shifted to 31547 Da, −584 Da which equals the molecular weight of two NeuAc residues (582.6 Da), and further shifted to 31346 Da, −201 Da (HexNAc) after HEXase II treatment. Theoretically, the lost HexNAc residue may be assigned to GalNAc or GlcNAc. But since monosaccharide GalNAc was found and the GalNAc was linked to GlcNAc, the lost HexNAc is GalNAc and not GlcNAc. Hence, the oligosaccharide for 32131 Da mass is (NeuAc)2(Gal)1(GalNAc)1(GlcNAc)4(Man)3(Fuc)1.

Based on these experimental results, the FLINT primary glycoforms were assigned as mono- and disialylated diantennary oligosaccharides with zero to two N-acetylgalactosamine residues, which replace galactose residues in common diantennary structures. Most of the oligosaccharides found contain Fuc residues in the core structure of the pentasaccharide. Small amounts of triantennary, sialo diantennary and sulfated oligosaccharides were also found.

The fluorescence labeled FLINT oligosaccharides were fractionated into multiple peaks on WAE HPLC according to their negative charge number. The primary structures of identified oligosaccharides in each fraction are mono and di-sialylated diantennary oligosaccharides, which account for over 70% of the total oligosaccharides. Tri- and/or tetra-antennary oligosaccharides were less than 10% of the total carbohydrate.

When the protein is fully glycosylated, the molar ratio of the sialic acid in the protein can be estimated using the normalized peak area ratio of the fluorescence labeled oligosaccharides in the WEA HPLC chromatogram.

Because FLINT contains only one glycosylation site that was fully glycosylated (Non-glycosylated protein was not detected by LC/MS.), sialic acid content can be calculated based on the results of WEA HPLC as follows:

Sialic Acid Content=Σsialylation degree of fraction×percentage of fraction/100

The average sialic acid content of this preparation of FLINT is estimated to be approximately 1.5 mole per mole protein using this fluorescence labeling approach. Similarly, the sialic acid to protein ratio could also be determined by HPLC/MS data. Applying the same strategy, the molar ratio of sialic acid to protein was determined to be 1.4 for LC/MS analysis. The advantage of using the normalized peak areas in the WEA HPLC chromatogram to calculate the sialic acid content is that the exact amount of FLINT does not need to be accurately determined.

Sialic acid content may also be determined by a modification of the procedure of Jourdian et al. J. Biol. Chem. 246, 430 (1971). The sialic acid residues are cleaved from glycoproteins by hydrolysis with 0.35M sulfuric acid at 80° C. for 30 minutes and the solutions neutralized with sodium hydroxide prior to analysis. In order to estimate the amount of protein present, a Bradford protein assay (Bradford Anal. Biochem. 72, 248 (1976)) using recombinant FLINT as standard is performed using the assay reagents and the micro-method procedure supplied by Bio-Rad.

### EXAMPLE 3

### Effect of Higher Sialylation on Clearance of R218Q FLINT

The pharmacokinetics of two lots (C7T-63A-1 and C7Z-PBS-32) of FLINT(R21Q) were examined for the influence of sialic acid content on the clearance in male Cynomolgus monkeys. Based on LC/MS and oligosaccharide profiling, lot#C7T-63A-1 had a sialic acid ratio of 1.7 while lot#C7Z-PBS-32 had a sialic acid ratio of 0.4.

Lots of FLINT(R218Q) were administered as a single intravenous bolus dose (0.5 mg/kg) and blood samples were obtained over a 48 h period after dosing.

Plasma samples from treated animals were analyzed for concentrations of FLINT(R218Q) using a sandwich ELISA method employing affinity purified rabbit polyclonal anti-FLINT antibodies. The capture antibody recognizes the N-terminal portion of FLINT(R218Q). The sandwich antibody was a biotinylated polyclonal antibody which recognizes the C-terminal portion of FLINT(R218Q). As formatted, measurement in the ELISA relies on the presence of the full length FLINT(R218Q), or molecules with minimal degradation at the N— or C-terminal regions.

After intravenous administration, both lots of FLINT(R218Q) were cleared from the plasma in a bi-phasic manner. The clearance of both lots of compound was dominated by a rapid distribution phase. Overall, the systemic clearance of FLINT(R218Q) from lot C7Z-PBS-32 (sialic acid ratio: 0.4) was approximately 2.5-fold faster than that of lot C7T-63A-1 (sialic acid ratio: 1.7).

The initial distribution of FLINT(R218Q) from lot C7Z-PBS-32 was more extensive than observed with the more fully sialated form (lot C7T-63A-1). Approximately 12% and 23% of the 5 min levels remained in the plasma 1 hour after administration of FLINT(R218Q) from lots C7z-PBS-32 and C7T-63A-1, respectively. In addition, concentrations at the first time point (5 min) were lower after administration of lot C7Z-PBS-32 (5.48 μg/ml) than after administration of lot C7T-63A-1 (8.47 μg/ml). The terminal phase t_{1/2} was somewhat shorter for FLINT(R218Q) having a lower sialic acid content. (8.3 vs. 12.7 hr; Table 1). The data indicate that the extent of terminal sialylation on the carbohydrate moieties present on FLINT(R218Q) have an influence on the clearance kinetics of the compound after administration by the intravenous route. The increased rate of clearance of the poorly sialylated molecules from the circulation is most likely mediated through hepatic asialoglycoprotein receptors via exposed terminal galactose residues.

### EXAMPLE 4

### Effect of Higher Sialylation on Clearance of Native FLINT

The pharmacokinetics of multiple lots FLINT are examined for the influence of sialic acid content on the clearance of FLINT in mammals, for example, male Cynomolgus monkeys. Oligosaccharide and sialic acid profiling using LC-MS is used to ascertain sialylation differences between lots.

Lots of FLINT are administered as a single intravenous bolus dose to Cynomolgus monkeys (0.5 mg/kg), and blood samples are obtained over a 48 hour period after dosing.

Plasma samples from treated animals are analyzed for concentrations of FLINT using a sandwich ELISA method employing affinity purified rabbit polyclonal anti-FLINT antibodies. The capture antibody recognizes the N-terminal portion of FLINT. The sandwich antibody is a biotinylated polyclonal antibody which recognizes the C-terminal portion of FLINT. As formatted, measurement in the ELISA relies on the presence of the full length FLINT, or molecules with minimal degradation at the N— or C-terminal regions.

The effect of sialylation levels on the clearance kinetics of FLINT is determined as in Example 3.

### EXAMPLE 5

### Fractionation of Recombinant FLINT Isoforms Using a Low pH Gradient on SP-Sepharose

FLINT isoforms are separated using a gradient of decreasing pH and increasing ionic strength. Concentrated and filtered FLINT-containing medium is loaded onto a column of SP-Sepharose at a ratio of approximately 20 mg total protein/mL gel. The column is then washed with approximately three column volumes of 20 mM MOPS, pH 5.5. FLINT isoforms are eluted from the column using a gradient starting with 20 mM MOPS, pH 5.5 and running to 20 mM MOPS, 600 mM NaCl, pH 5.5. The total volume of the gradient is approximately 40 column volumes.

While the invention has been described in what is considered to be its preferred embodiments, it is not to be limited to the disclosed embodiments, but on the contrary, is intended to cover various modifications and equivalents included within the spirit and scope of the appended claims, which scope is to be accorded the broadest interpretation so as to encompass all such modifications and equivalents.

We claim:

1. A composition comprising FLINT isoforms said composition having an average sialic acid content of less than about 0.5 sialic acids per molecule of FLINT.

2. A composition comprising FLINT isoforms said composition having an average sialic acid content of about 0.5 sialic acids per molecule of FLINT.

3. A composition comprising FLINT isoforms said composition having an average sialic acid content of about 1.0 sialic acids per molecule of FLINT.

4. A composition comprising FLINT isoforms said composition having an average sialic acid content of about 1.5 sialic acids per molecule of FLINT.

5. A composition comprising FLINT isoforms said composition having an average sialic acid content of about 2.0 sialic acids per molecule of FLINT.

6. A composition comprising FLINT isoforms said composition having an average sialic acid content of about 2.5 sialic acids per molecule of FLINT.

7. A composition comprising FLINT isoforms said composition having an average sialic acid content of about 3.0 sialic acids per molecule of FLINT.

8. A composition comprising FLINT isoforms said composition having an average sialic acid content of about 3.5 sialic acids per molecule of FLINT.

9. A composition comprising FLINT isoforms said composition having an average sialic acid content of about 4.0 sialic acids per molecule of FLINT.

10. A composition comprising FLINT isoforms said composition having an average sialic acid content of about 4.5 sialic acids per molecule of FLINT.

11. A composition comprising FLINT isoforms said composition having an average sialic acid content of about 5.0 sialic acids per molecule of FLINT.

12. A composition comprising FLINT isoforms said composition having an average sialic acid content of about 5.5 sialic acids per molecule of FLINT.

13. A composition comprising FLINT isoforms said composition having an average sialic acid content of about 6.0 sialic acids per molecule of FLINT.

14. A composition as in any one of claims 1 to 13 wherein said isoform is protease resistant FLINT analog R218Q.

15. A pharmaceutical composition comprising a therapeutically effective amount of a FLINT isoform and a pharmaceutically acceptable diluent, adjuvant or carrier.

16. A composition as in claim 15 wherein said FLINT is R218Q.
